(19) United States
(12) Patent Application Publication
(10) Pub. No.: US 2022/0323114 A1
(43) Pub. Date: Oct. 13, 2022

(54) SPINAL STABILIZATION APPARATUS FOR THE DETECTION AND PREVENTION OF A LOSS OF ROBOTIC ACCURACY AND METHOD THEREOF

(71) Applicant: Ernest Wright, Nashville, TN (US)

(72) Inventor: Ernest Wright, Nashville, TN (US)

(21) Appl. No.: 17/301,558

(22) Filed: Apr. 7, 2021

(51) Int. Cl. A61B 17/70; A61B 34/30; A61B 90/00

(52) U.S. Cl. CPC A61B 17/7013; A61B 34/30; A61B 90/08; A61B 90/39; A61B 2090/0811; A61B 2090/3983

(57) ABSTRACT

A spinal stabilization apparatus and method thereof for preventing and detecting the unintended movement of the spine of a patient during spinal surgery performed at least in part by a robot is disclosed herein. The spinal stabilization apparatus may comprise a proximal attachment member, a distal attachment member, a first adjustable attachment arm, and a second adjustable attachment arm, defining a frame, configured to stabilize the spine of the patient. For detecting and preventing unintended movement of the spine, the spinal stabilization apparatus may further include optical reference spheres and at least one strain gauge. The optical reference spheres are coupled to the frame and the spatial relationship therebetween may be monitored by the robot. The at least one strain gauge may be incorporated into the connection between frame and the robot and allows for monitoring of the amount of force being applied to the patient.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND

### 1. Field of the Invention

The present disclosure relates generally to robotic spinal surgery.

More particularly, this disclosure pertains to a stabilization apparatus for the detection and prevention of a loss of accuracy in robotic spinal surgery.

### 2. Description of the Prior Art

The use of robots in many surgical disciplines has become commonplace, yet a role for robots in spine surgery remained elusive due to the mobility of the spinal column and its intimate relationship with the spinal cord and nerve roots. The proximity of critical neural structures to the vertebrae necessitates millimetric accuracy, which has been difficult to achieve with the required consistency for practical clinical implementation.

While the first FDA approved spine robot was introduced in 2004, the use of robots in spine surgery remains limited. These robots represent the first generation of practically useful spinal robots, with their sole function being the accurate placement of implants into the bony spinal column.

There are, however, some fundamental challenges which must be overcome to grant the ease of use and dependability that will allow for widespread adoption of the robots in spinal surgery.

It is important to understand that these robots do not currently have the technology to track the location of the patient's spinal anatomy in real time. Consequently, at the beginning of surgery the robot is “registered” or familiarized with the patient's anatomy before surgery begins. Once this registration has been obtained, the robot will assume that the relationship between the robot and the patient's anatomy will not change for the duration of the surgery. As such, it is critical that the patient's spine not move in order to preserve the accuracy of the robot.

Foremost among the weaknesses of spinal robots is a loss of spatial accuracy leading to misplacement of implanted hardware in a patient's spine. Substantial resources have been dedicated to teaching the spinal robot how to match a 3D CT scan of the spine with the patient's spine on the operating room table, and this can now be done consistently with the required accuracy. Retaining that initial accuracy over the course of an entire surgical procedure, during which the spinal column is being heavily manipulated, remains an ongoing challenge.

Currently, spinal robots are either connected to the patient via a rigid attachment to the pelvis or function completely independently without any physical attachment to the patient's anatomy. Both of these operational paradigms have shortcomings: because the spine is articulated, it can move substantially relative to the pelvis making this method of attachment prone to an undetected loss of accuracy. In a system without any physical attachment at all, there is a complete reliance on optical navigation to detect a loss of accuracy without any physical attachment to detect or prevent such an error.

The maintenance of navigational accuracy is a seminal challenge in the development of spinal robotics. Detecting and preventing patient movement as a source of inaccuracy is a fundamental and unsolved challenge to establishing the dependability necessary for the widespread adoption of robotics in spine surgery.

### BRIEF SUMMARY

In view of at least some of the above-referenced problems in conventional spinal surgery performed at least in part by a robot, an exemplary object of the present disclosure may be to provide a spinal stabilization apparatus that is configured to stabilize, detect, and prevent movement of the spine of a patient during spinal surgery.

An exemplary such apparatus may feature a three-point attachment frame, which may be affixed to the patient at both the proximal and distal ends of the spine to firmly triangulate the spinal column. The exemplary such apparatus may be affixed to the robot via a rigid connection. The exemplary such apparatus presents a straightforward solution prevents shifting of the spine in all three axes of space (e.g., x-, y-, and z-axes), thereby maintaining registration throughout surgery and preventing a loss of navigational accuracy. The exemplary such apparatus may further feature a strain gauge incorporated into the connection between the frame and the robot that allows for monitoring of the amount of force being applied to the patient. The robot (or an external device) may be programmed to activate an alarm state in the robot (or the external device) to thereby notify the surgeon of an impending loss of accuracy, and therefore prevent a potential catastrophic patient outcome.

The exemplary such apparatus may further feature a plurality of optical reference points (e.g., reflective spheres) positionable along the frame (e.g., at articulating joints of the frame). The reference spheres will piggyback on the robot's “optical navigation system” such that the robot will localize these references spheres in 3-dimensional space. Should there by a shift in the frame, the reference spheres will move relative to each other and the robot may be configured to immediately notify the surgeon that the has been a frame shift and a loss of accuracy.

In a particular embodiment, an exemplary spinal stabilization apparatus for preventing and detecting the unintended movement of a spine of a patient during spinal surgery performed at least partially by a robot is disclosed herein. The spinal stabilization apparatus may comprise a proximal attachment member, a distal attachment member, a first adjustable attachment arm, and a second adjustable attachment arm. The proximal attachment member may be configured to be positioned above a sacrum bone of the patient. The distal attachment member may be configured to be coupled to at least one vertebra of the spine of the patient. The first adjustable attachment arm may be configured to be coupled between the proximal attachment member and the distal attachment member. The first adjustable attachment arm may further be configured to be coupled to a first point on the pelvis or sacrum of the patient. The second adjustable attachment arm may be configured to be coupled between the proximal attachment member and the distal attachment member and extend away from the first adjustable attachment arm. The second adjustable attachment arm may further be configured to be coupled to a second point on the pelvis or sacrum of the patient.

In an exemplary aspect according to the above-referenced embodiment, the first and second adjustable attachment arms may be positioned bilaterally symmetrically or asymmetrically about the spine of the patient when the spinal stabilization apparatus is attached to the spine of the patient

In another exemplary aspect according to the above-referenced embodiment, the distal attachment member may be offset from the proximal attachment member by an offset distance. In accordance with this aspect, each of the first and second adjustable attachment arms may be longer than the offset distance.

In another exemplary aspect according to the above-referenced embodiment, the spinal stabilization apparatus may further comprise a first threaded post and a second threaded post. The first threaded post may be configured to couple the first adjustable attachment arm to an ilium portion of the first hip bone of the patient. The second threaded post may be configured to couple the second adjustable attachment arm to an ilium portion of the second hip bone of the patient.

In another exemplary aspect according to the above-referenced embodiment, the first threaded post may include a first end configured to be attached to the ilium portion of the first hip bone of the patient and a second end configured to be attached to the first adjustable attachment arm. In accordance with this aspect, the second threaded post may include a first end configured to be attached to the ilium portion of the second hip bone of the patient and a second end configured to be attached to the second adjustable attachment arm.

In another exemplary aspect according to the above-referenced embodiment, the first adjustable attachment arm may include a first arm proximal member, a first arm intermediate member, and a first arm distal member pivotally coupled together. The first arm proximal member may be configured to be coupled to the proximal attachment member. The first arm distal member may be configured to be coupled to the distal attachment member. In accordance with this aspect, the second adjustable attachment arm may include a second arm proximal member, a second arm intermediate member, and a second arm distal member pivotally coupled together. The second arm proximal member may be configured to be coupled to the proximal attachment member. The second arm distal member may be configured to be coupled to the distal attachment member.

In another exemplary aspect according to the above-referenced embodiment, the first arm intermediate member may be coupled between free ends of the first arm proximal member and the first arm distal member; and the second arm intermediate member may be coupled between free ends of the second arm proximal member and the second arm distal member

In another exemplary aspect according to the above-referenced embodiment, the first arm proximal member may include a first arm proximal member first end and a first arm proximal member second end. The first arm proximal member may be configured to be coupled to a first threaded post of the spinal stabilization apparatus between the first arm proximal member first end and the first arm proximal member second end. The first threaded post may be configured to be coupled to an ilium portion of the first hip bone of the patient. In accordance with this aspect, the second arm proximal member may include a second arm proximal member first end and a second arm proximal member second end. The second arm proximal member may be configured to be coupled to a second threaded post of the spinal stabilization apparatus between the second arm proximal member first end and the second arm proximal member second end. The second threaded post may be configured to be coupled to an ilium portion of the second hip bone of the patient.

In another exemplary aspect according to the above-referenced embodiment, the spinal stabilization apparatus may further comprise a plurality of threaded optical reference spheres. In accordance with this aspect, one threaded optical reference sphere of the plurality of threaded optical reference spheres may be positioned at each end of each of the first arm proximal member, the first arm distal member, the second arm proximal member, and the second arm distal member.

In another exemplary aspect according to the above-referenced embodiment, the first and second arm intermediate members may be telescopic in length.

In another exemplary aspect according to the above-referenced embodiment, each of the first arm proximal member, the first arm intermediate member, and the first arm distal member may be configured to be pivotally coupled together using a first plurality of fasteners and each of the second arm proximal member, the second arm intermediate member, and the second arm distal member may be configured to be pivotally coupled together using a second plurality of fasteners.

In another exemplary aspect according to the above-referenced embodiment, the spinal stabilization apparatus may further comprise a plurality of threaded optical reference spheres coupled along each of the first and second adjustable attachment arms. In accordance with this aspect, the plurality of threaded optical reference spheres may be configured to be monitored for determining if the spine of the patient moves during the course of the spinal surgery

In another exemplary aspect according to the above-referenced embodiment, the distal attachment member may be a spinous process clamp including a vertical post. The vertical post may be configured to couple to each of the first and second adjustable attachment arms.

In another exemplary aspect according to the above-referenced embodiment, the proximal attachment member may include a proximal robot attachment portion and the distal attachment member may include a distal robot attachment portion. In accordance with this aspect, one or more of the proximal robot attachment portion or the distal robot attachment portion may be configured to couple to a portion of the robot.

In another exemplary aspect according to the above-referenced embodiment, the spinal stabilization apparatus may further comprise at least one strain gauge configured to couple to one or more of the proximal robot attachment portion or the distal robot attachment portion. In accordance with this aspect, the at least one strain gauge may be configured to monitor sprain applied to the spinal stabilization apparatus by at least the robot such that impending movement of the spine of the patient is identifiable.

In another embodiment, a method of stabilizing a spine of a patient with a spinal stabilization apparatus for spinal surgery performed at least in part by a robot is disclosed herein. The method may comprise (a) coupling at least one threaded post of the spinal stabilization apparatus to one of an ilium portion of a hip bone or a sacrum bone of the patient; (b) coupling a clamp of the spinal stabilization apparatus to a spinous process of a vertebra of the spine of the patient; (c) positioning a proximal attachment member of the spinal stabilization apparatus above the sacrum bone of the patient; and (d) attaching first and second adjustable attachment arms of the spinal stabilization apparatus between the at least one threaded post and the clamp such that each of the first and second adjustable attachment arms extend away from each other on opposite sides of the spine of the patient.

In an exemplary aspect according to the above-referenced embodiment, step (d) of the method may further comprise coupling each of the first and second adjustable attachment arms to the proximal attachment member.

In another exemplary aspect according to the above-referenced embodiment, step (d) of the method may further comprise adjusting a positioning of a plurality of arm members of each of the first and second adjustable attachment arms to surround an area of surgical interest; and rigidly coupling the plurality of arm members of each of the first and second adjustable attachment arms together to stabilize at least a portion of the spine of the patient positioned between the clamp and the proximal attachment member and highlighted by the area of surgical interest.

In another exemplary aspect according to the above-referenced embodiment, the method may further comprise coupling a plurality of optical reference spheres to each of the first and second adjustable attachment arms such that one optical reference sphere of the plurality of optical reference spheres is positioned at each end of the plurality of arm members of the first and second adjustable attachment arms.

In another exemplary aspect according to the above-referenced embodiment, the method may further comprise coupling a plurality of optical reference spheres along each of the first and second adjustable attachment arms

### BRIEF DESCRIPTION OF THE SEVERAL VIEWS OF THE DRAWINGS

FIG. 1 is a top plan view of a spinal stabilization apparatus coupled to a spine of a patient in accordance with the present disclosure.

FIG. 2 is a side elevation view of the spinal stabilization apparatus of FIG. 1 coupled to the spine of the patient in accordance with the present disclosure.

FIG. 3 is a perspective view of the spinal stabilization apparatus of FIG. 1 in accordance with the present disclosure.

FIG. 4A is an exploded perspective view of the spinal stabilization apparatus of FIG. 3 including an embodiment of fasteners of the spinal stabilization apparatus in accordance with the present disclosure.

FIG. 4B is an exploded perspective view of the spinal stabilization apparatus of FIG. 3 including a different embodiment of fasteners of the spinal stabilization apparatus in accordance with the present disclosure.

FIG. 5A is a perspective view of an intermediate arm member of an adjustable attachment arm of the spinal stabilization apparatus of FIG. 3 in accordance with the present disclosure.

FIG. 5B is an exploded perspective view of the intermediate arm member of FIG. 5A in accordance with the present disclosure.

FIG. 6 is a front elevation view of a distal attachment member of the spinal stabilization apparatus of FIG. 1 in accordance with the present disclosure

FIG. 7 is a perspective view of a plurality of optical reference spheres of the spinal stabilization apparatus of FIG. 1 in accordance with the present disclosure.

FIG. 8 is a flow chart of a method of stabilizing the spine of the patient using the spinal stabilization apparatus of FIG. 1 in accordance with the present disclosure.

### DETAILED DESCRIPTION

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure.

The words “connected”, “attached”, “joined”, “mounted”, “fastened”, and the like should be interpreted to mean any manner of joining two objects including, but not limited to, the use of any fasteners such as screws, nuts and bolts, bolts, pin and clevis, and the like allowing for a stationary, translatable, or pivotable relationship; welding of any kind such as traditional MIG welding, TIG welding, friction welding, brazing, soldering, ultrasonic welding, torch welding, inductive welding, and the like; using any resin, glue, epoxy, and the like; being integrally formed as a single part together; any mechanical fit such as a friction fit, interference fit, slidable fit, rotatable fit, pivotable fit, and the like; any combination thereof; and the like.

Unless specifically stated otherwise, any part of the apparatus of the present disclosure may be made of any appropriate or suitable material including, but not limited to, metal, alloy, polymer, polymer mixture, wood, composite, or any combination thereof.

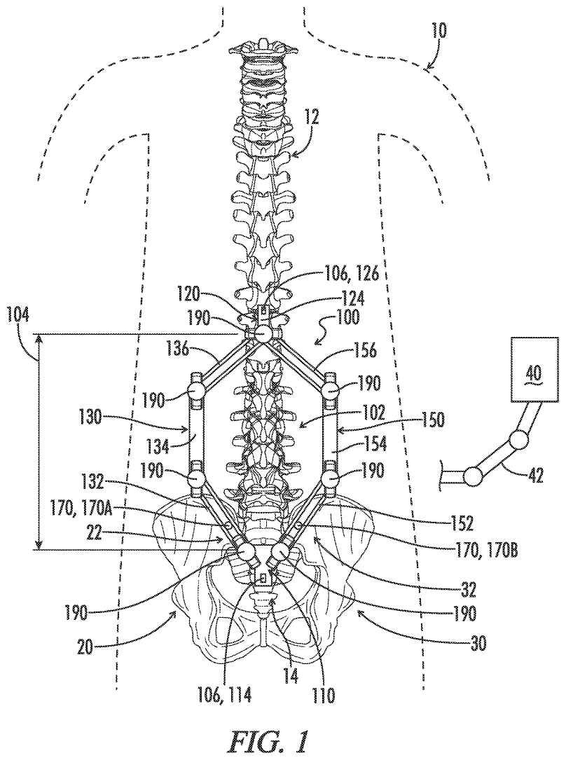

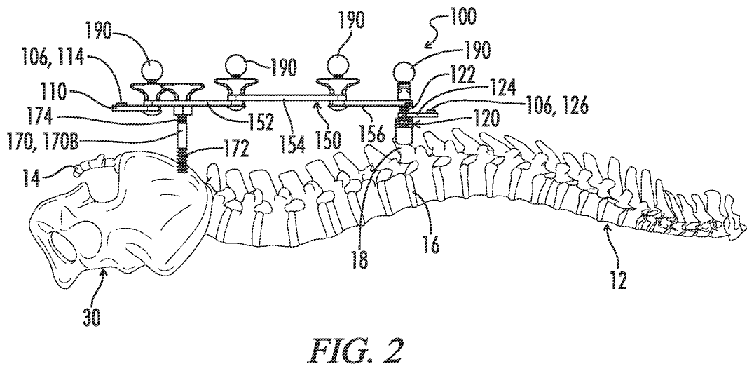

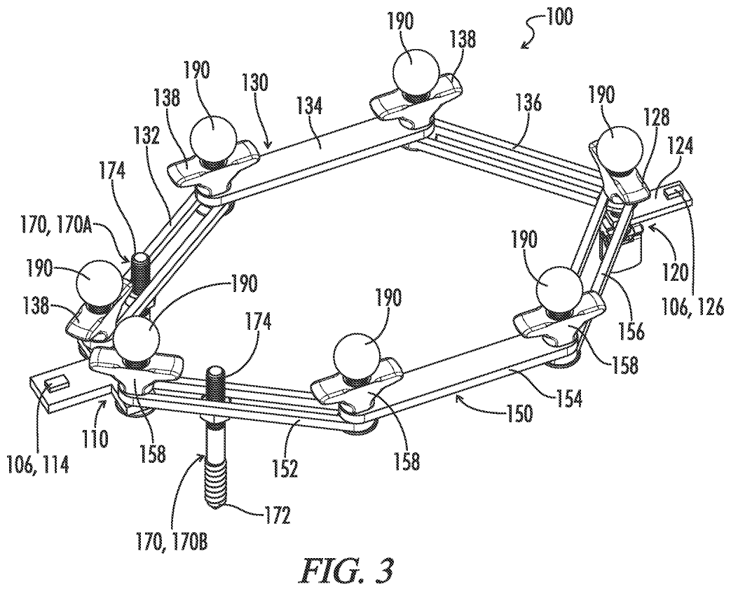

Referring to FIGS. 1-4, a spinal stabilization apparatus 100 is shown. The spinal stabilization apparatus 100 is configured coupled to a spine 12 of a patient 10 in order to stabilize and prevent movement of the spine 12 of the patient 10 during spinal surgery, which may be performed at least in part by a robot 40. More particularly, the spinal stabilization apparatus 100 may be configured to surround an area of surgical interest 102 of the spine 12 of the patient 10 and may further be configured to stabilize at least a portion of the spine 12 of the patient 10 highlighted by the area of surgical interest 102. The robot 40 may also be referred to herein as a surgical robot 40 or a spinal surgery robot 40. The robot 40 includes at least an articulating arm 42.

The spinal stabilization apparatus 100 may comprise a proximal attachment member 110, a distal attachment member 120, a first adjustable attachment arm 130, and a second adjustable attachment arm 150. The first and second adjustable attachment arms 130, 150 are configured to be attached between the proximal attachment member 110 and the distal attachment member 120 and are further configured to extend away from each other, for example, on opposite sides of the spine 12 of the patient 10 so as to not interfere with access to the spine 12 during surgery. The first and second adjustable attachment arms 130, 150 may surround the area of surgical interest 102 of the spine 12 of the patient 10 between the proximal attachment member 110 and the distal attachment member 120. In certain optional embodiments, the first and second adjustable attachment arms 130, 150 may be bilaterally symmetric about the spine 12 of the patient 10 when the spinal stabilization apparatus 100 is attached to the spine 12 of the patient 10.

The proximal attachment member 110 may be configured to be positioned above a sacrum bone 14 of the patient 10. The proximal attachment member 110 may include at least one arm attachment point 112. As illustrated, the at least one arm attachment point 112 includes a first arm attachment point 112A configured to be coupled to the first adjustable attachment arm 130 and a second arm attachment point 112B configured to be coupled to the second adjustable attachment arm 150. The at least one arm attachment point 112 may be a post, for example, a threaded post or the like. The first and second adjustable attachment arms 130, 150 may be coupled to the first and second arm attachment points 112A, 112B using fasteners as disclosed herein.

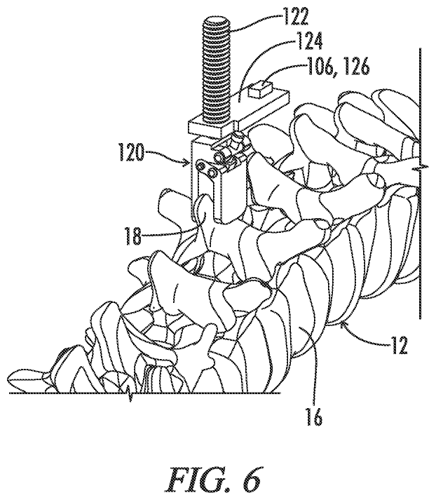

The distal attachment member 120 may be configured to be coupled to a spinous process 18 of a vertebra 16 of the patient 10. In certain optional embodiments (not shown), the distal attachment member 120 may be coupled to the spinous process of consecutive vertebrae. The distal attachment member 120 may also be referred to herein as a clamp 120. The distal attachment member 120 may be, for example, a spinous process clamp, as shown in FIG. 6. The distal attachment member 120 may include a vertical post 122 configured to be coupled to both the first adjustable attachment arm 130 and the second adjustable attachment arm 150. The vertical post 122 may include a threaded portion (not shown) for engaging a fastener 128 as disclosed herein. The fastener 128 may be substantially similar to the first and second pluralities of fasteners 138, 158.

The distal attachment member 120 may be offset from the proximal attachment member 110 by an offset distance 104. Each of the first and second adjustable attachment arms 130, 150 may be longer than the offset distance 104.

The spinal stabilization apparatus 100 may further include at least one threaded post 170 configured to be coupled to one of the sacrum bone 14, a first hip bone 20 of the patient 10, or a second hip bone 30 of the patient 10. The first hip bone 20 may also be referred to herein as a left hip bone 20 and the second hip bone 30 may also be referred to herein as a right hip bone 30. As illustrated in FIG. 1, the at least one threaded post 170 includes a first threaded post 170A configured to be coupled to an ilium portion 22 of the first hip bone 20 and a second threaded post 170B configured to be coupled to an ilium portion 32 of the second hip bone 30. More specifically, the first threaded post 170A may be coupled to the posterior superior iliac spine of the ilium portion 22 of the first hip bone 20 and the second threaded post 170B may be coupled to the posterior superior iliac spine of the ilium portion 32 of the second hip bone 30.

In certain optional embodiments, the first adjustable attachment arm 130 may be configured to be coupled to ilium portion 22 of the first hip bone 20 of the patient 10 using, for example, the first threaded post 170A. Likewise, the second adjustable attachment arm 150 may be configured to be coupled to ilium portion 32 of the second hip bone 30 of the patient 10 using, for example, the second threaded post 170B. In other optional embodiments (not shown), the at least one threaded post 170 may be coupled between the proximal attachment member 110 and the sacrum bone 14 of the patient 10.

Each of the at least one threaded post 170 may include a first end 172 and a second end 174. The first end 172 may be configured to be attached to one of the sacrum bone 14, the ilium portion 22 of the first hip bone 20, or the ilium portion 32 of the second hip bone 30. The second end 174 may be configured to be coupled to one of the proximal attachment member 110, the first adjustable attachment arm 130, or the second adjustable attachment arm 150.

The first adjustable attachment arm 130 may include a first arm proximal member 132, a first arm intermediate member 134, and a first arm distal member 136 configured to be pivotally coupled together. The first arm proximal member 132 may be configured to be coupled to the proximal attachment member 110. The first arm distal member 136 may be configured to be coupled to the distal attachment member 120. The first arm intermediate member 134 may be configured to be coupled between the first arm proximal member 132 and the first arm distal member 136.

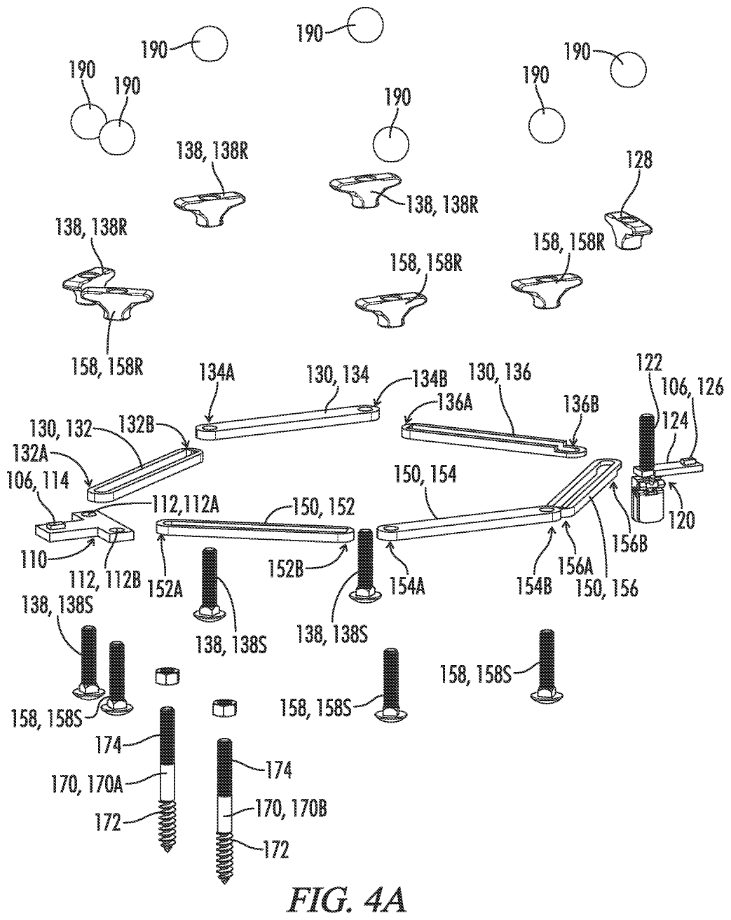

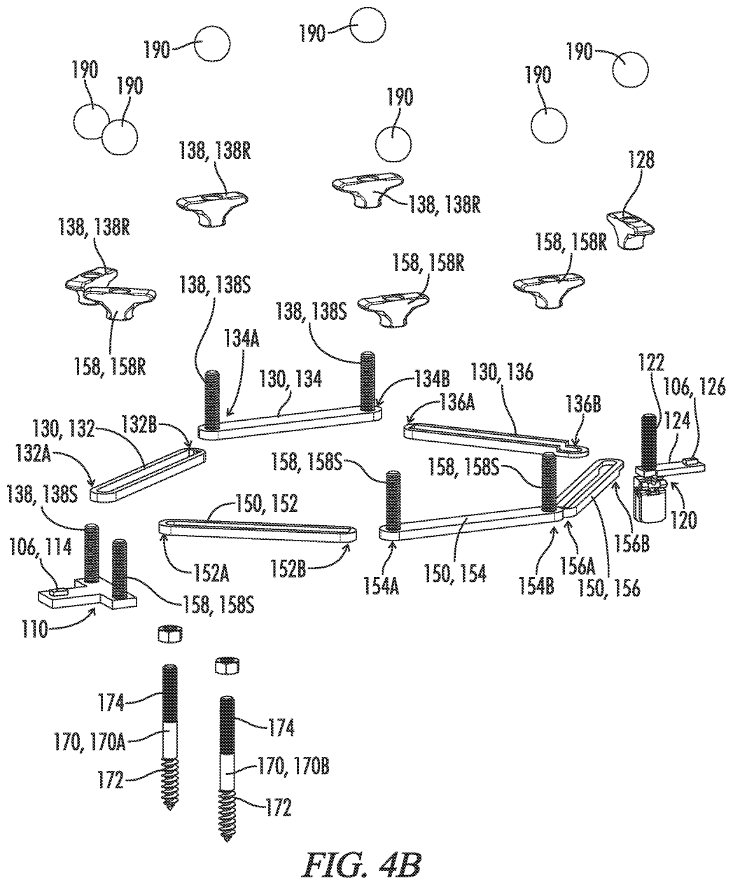

Referring to FIG. 4, the first arm proximal member 132 may include a first arm proximal member first end 132A and a first arm proximal member second end 132B. The first arm intermediate member 134 may include a first arm intermediate member first end 134A and a first arm intermediate member second end 134B. The first arm distal member 136 may include a first arm distal member first end 136A and a first arm distal member second end 136B. The first arm proximal member first end 132A may be configured to be coupled to the proximal attachment member 110. The first arm intermediate member first end 134A may be configured to be coupled to the first arm proximal member second end 132B and the first arm intermediate member second end 134B may be configured to be coupled to the first arm distal member first end 136A. The first arm distal member second end 136B may be configured to be coupled to the distal attachment member 120.

The couplings between the first arm proximal member 132, the first arm intermediate member 134, and the first arm distal member 136 may be defined by a first plurality of fasteners 138. The first plurality of fasteners 138 may comprise an exteriorly threaded shaft 138S and an interiorly treaded receiver 138R. The exteriorly threaded shaft 138S may comprise, for example, a threaded fastener such as a bolt, an all-thread shaft, or the like. As illustrated in FIG. 4A, the exteriorly threaded shaft 138S may be a bolt that is separable from each of the first arm proximal member 132, the first arm intermediate member 134, and the first arm distal member 136. As illustrated in FIG. 4B, the exteriorly threaded shaft 138S may be an all-thread shaft attached to and extending from each of the first arm proximal member second end 132B and the first arm distal member first end 136A. The interiorly treaded receiver 138R may comprise, for example, a nut, a wing nut, a hand/thru knob (shown in FIG. 4A), a cam handle, or the like. Once the positions of the first arm proximal member 132, the first arm intermediate member 134, and the first arm distal member 136 are set, the first plurality of fasteners 138 may be utilized to fix their relative positions using frictional engagement. In certain optional embodiments, a rubber washer (not shown) may be positioned between the members of the first adjustable arm 130 for increasing the frictional engagement and preventing movement.

The second adjustable attachment arm 150 may include a second arm proximal member 152, a second arm intermediate member 154, and a second arm distal member 156 configured to be pivotally coupled together. The second arm proximal member 152 may be configured to be coupled to the proximal attachment member 110. The second arm distal member 156 may be configured to be coupled to the distal attachment member 120. The second arm intermediate member 154 may be configured to be coupled between the second arm proximal member 152 and the second arm distal member 156.

Referring to FIG. 4, the second arm proximal member 152 may include a second arm proximal member first end 152A and a second arm proximal member second end 152B. The second arm intermediate member 154 may include a second arm intermediate member first end 154A and a second arm intermediate member second end 154B. The second arm distal member 156 may include a second arm distal member first end 156A and a second arm distal member second end 156B. The second arm proximal member first end 152A may be configured to be coupled to the proximal attachment member 110. The second arm intermediate member first end 154A may be configured to be coupled to the second arm proximal member second end 152B and the second arm intermediate member second end 154B may be configured to be coupled to the second arm distal member first end 156A. The second arm distal member second end 156B may be configured to be coupled to the distal attachment member 120.

The couplings between the second arm proximal member 152, the second arm intermediate member 154, and the second arm distal member 156 may be accomplished using a second plurality of fasteners 158. The second plurality of fasteners 158 may be identical to the first plurality of fasteners 138. The second plurality of fasteners 158 may comprise an exteriorly threaded shaft 158S and an interiorly treaded receiver 158R. The exteriorly threaded shaft 158S may comprise, for example, a threaded fastener such as a bolt, an all-thread shaft, or the like. As illustrated in FIG. 4A, the exteriorly threaded shaft 158S may be a bolt that is separable from each of the second arm proximal member 152, the second arm intermediate member 154, and the second arm distal member 156. As illustrated in FIG. 4B, the exteriorly threaded shaft 158S may be an all-thread shaft attached to and extending from each of the second arm proximal member second end 152B and the second arm distal member first end 156A. The interiorly treaded receiver 158R may comprise, for example, a nut, a wing nut, a hand/thru knob (shown in FIG. 4A), a cam handle, or the like. Once the positions of the second arm proximal member 152, the second arm intermediate member 154, and the second arm distal member 156 are set, the second plurality of fasteners 158 may be utilized to fix their relative positions using frictional engagement. In certain optional embodiments, a rubber washer (not shown) may be positioned between the members of the second adjustable arm 150 for increasing the frictional engagement and preventing movement.

Each of the first arm proximal member 132 and the second arm proximal member 152 may be identical. Likewise, each of the first arm intermediate member 134 and the second arm intermediate member 154 may be identical. Finally, each of the first arm distal member 136 and the second arm distal member 156 may be identical.

The first threaded post 170A may be coupled to the first arm proximal member 132 between the first arm proximal member first end 132A and the first arm proximal member second end 132B. In certain optional embodiments, the first threaded post 170A may be coupled to the first arm proximal member 132 closer to the first arm proximal member first end 132A than to the first arm proximal member second end 132B. The second threaded post 170B may be coupled to the second arm proximal member 152 between the second arm proximal member first end 152A and the second arm proximal member second end 152B. In certain optional embodiments, the second threaded post 170B may be coupled to the second arm proximal member 152 closer to the second arm proximal member first end 152A than to the second arm proximal member second end 152B.

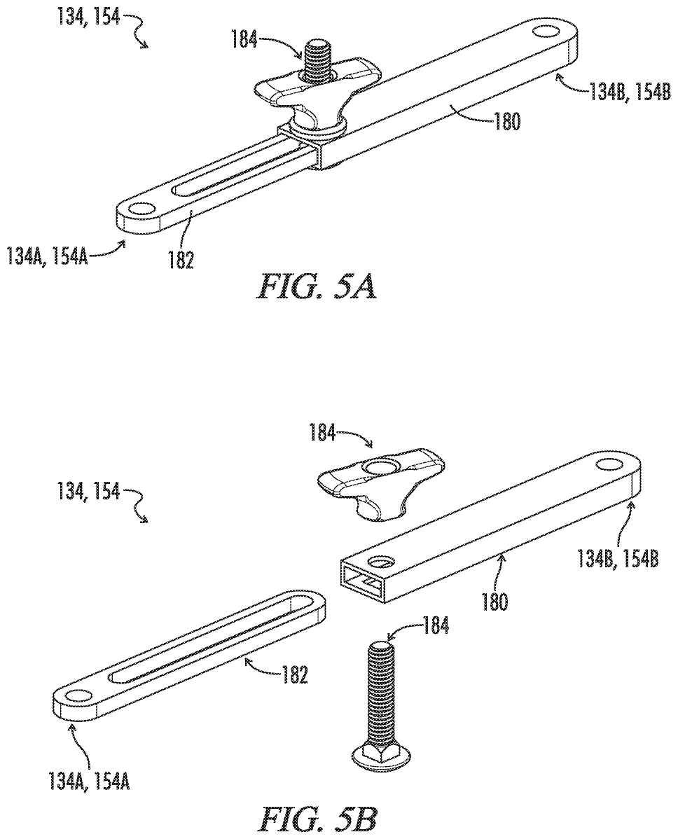

Referring to FIGS. 5A and 5B, each of the first arm intermediate member 134 and the second arm intermediate member 154 may be telescopic. Each of the first and second arm intermediate members 134, 154 may include a female portion 180 configured to slidably receive a male portion 182. Movement of the female potion 180 relative to the male portion 182 may be fixed using a fastener 184. The fastener 184 of each of the first and second arm intermediate members 134, 154 may be substantially similar to the first and second pluralities of fasteners 138, 158.

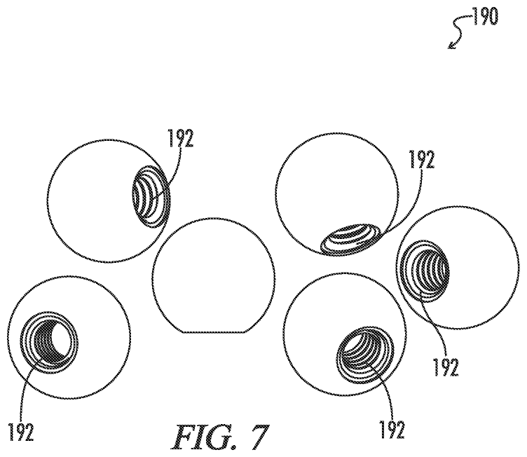

The spinal stabilization apparatus 100 may further include a plurality of threaded optical reference spheres 190 configured to be coupled to along each of the first and second adjustable attachment arms 130, 150. The plurality of threaded optical reference spheres 190 may also be referred to herein as a plurality of optical reference spheres 190. Referring to FIG. 7, each of the plurality of threaded optical reference spheres 190 may include a threaded insert 192. The plurality of threaded optical reference spheres 190 are configured to be monitored, for example, by the robot 40, for determining if the spine 12 of the patient 10 moves during the course of a surgery. Each of the plurality of threaded optical reference spheres 190 may be coupled to one of the vertical post 122 of the distal attachment member 120, the first and second attachment points 112A, 112B of the proximal attachment member 110, the exteriorly threaded shaft 138S at the first arm proximal member second end 132B, the exteriorly threaded shaft 158S at the second arm proximal member second end 152B, the exteriorly threaded shaft 138S at the first arm distal member first end 136A, or the exteriorly threaded shaft 158S at the second arm distal member first end 156A, via the threaded insert 192. Essentially, the plurality of threaded optical reference spheres 190 are configured to be coupled to a joint of each of the first and second adjustable attachment arms 130, 150. A joint of each of the first and second adjustable attachment arms 130, 150 may be defined at both ends of each of the first and second arm proximal members 132, 152 and the first and second distal arm members 136, 156.

The arm members (e.g., the first arm proximal, intermediate, and distal members 132, 134, 136 and the second arm proximal, intermediate, and distal members 152, 154, 156) of each of the first and second adjustable attachment arms 130, 150 may be constructed of a rigid substrate, such as, for example, a metal alloy, or alternatively a composite material which includes rigid properties while also having radiolucent properties which will prevent the arm members from obstructing intraoperative x-rays. The certain optional embodiments, the proximal and distal attachment members 110, 120 may also be constructed of the same or similar material to that of the arm members.

Referring to FIG. 1, the proximal attachment member 110 may include a proximal robot attachment portion 114. The distal attachment member 120 may include a robot interface platform 124 coupled to the vertical post 122. The robot interface platform 124 may include a distal robot attachment potion 126. One or more of the proximal robot attachment portion 114 or the distal robot attachment portion 126 may be configured to receive (or coupled to) a portion (e.g., a portion of an articulating arm or the like) of the robot 40 for fixing a position of the patient 10 and the spinal stabilization apparatus 100 relative to the robot 40.

The spinal stabilization apparatus 100 may further include at least one strain gauge 106 configured to couple to one or more of the proximal robot attachment portion 114 or the distal robot attachment portion 126. The at least one strain gauge 106 may be configured to monitor a strain applied to the spinal stabilization apparatus 100 by at least the robot 40 such that impending movement of the spine 12 of the patient 10 can be determined.

In certain optional embodiments, the robot 40 may be attached to both the proximal robot attachment portion 114 and the distal robot attachment portion 126. Each of the proximal robot attachment portion 114 and the distal robot attachment portion 126 may include an interlocking tab (e.g., male/female joint, not shown) secured by a screw or other fastening means. The at least one strain gauge 106 may be integrated into the base of this tab (not shown) to accurately measure the forces being placed on the patient 10 relative to the robot 40 to detect a potential loss of accuracy.

The at least one strain gauge 106 and the plurality of threaded optical reference spheres 190 are two structures of the spinal stabilization apparatus 100 which may be utilized, for example, by the robot 40 (or some other external device) to detect a loss of accuracy (e.g., movement or impending movement of the spine 12 of the patient 10).

The at least one strain gauge 106 of the spinal stabilization apparatus 100 may be configured to transmit (wirelessly or wired) a constant voltage output to the base station (not shown) of the robot 40. This not only allows for user defined thresholds for an alarm to sound at the computer base station should the threshold value be exceeded, but also allows for recording of data and retrospective data review in cases in which a loss of accuracy is subsequently discovered after the surgery has been completed. The ability to retrospectively parse through this force data from many surgical cases will allow physicians to better understand the critical thresholds of force beyond which accuracy is lost and better predict a loss of accuracy in the future.

The plurality of threaded optical reference spheres 190 of the spinal stabilization apparatus 100 may piggyback on an infrared optical navigation system (not shown) of the robot 40. The infrared optical navigation system is the basis for localizing the articulating arm 42 of the robot 40 in 3D space. The plurality of threaded optical reference spheres 190 of the spinal stabilization apparatus 100 may be utilized by the robot 40 such that the infrared optical navigation system of the robot 40 can visualize the location of each of the first and second adjustable attachment arms 130, 150 in 3D space. The navigation system may then visualize and monitor the relationship between the first and second adjustable attachment arms 130, 150 as a 3D shape (e.g., the area of surgical interest 102), and sound an alarm if the spatial relationship between the plurality of threaded optical reference spheres 190 changes to suggest that a joint of the spine 12 of the patient 10 has shifted.

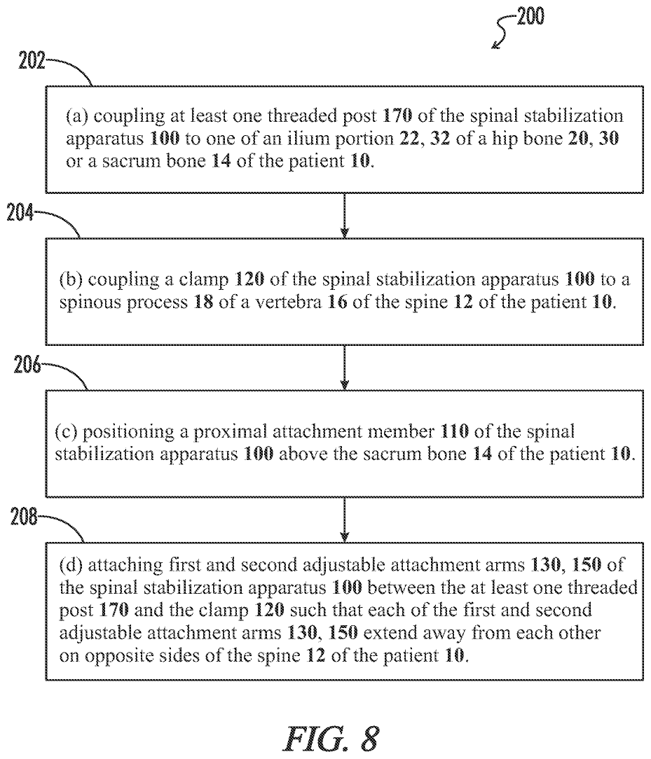

Referring to FIG. 8, a method 200 of stabilizing a spine 12 of a patient 10 with a spinal stabilization apparatus 100 for spinal surgery performed at least in part by a robot 40 is shown. The method 200 may comprise (a) coupling 202 at least one threaded post 170 of the spinal stabilization apparatus 100 to one of an ilium portion 22, 32 of a hip bone 20, 30 or a sacrum bone 14 of the patient 10. The method 200 may further comprise (b) coupling 204 a clamp 120 of the spinal stabilization apparatus 100 to a spinous process 18 of a vertebra 16 of the spine 12 of the patient 10. The method 200 may further comprise (c) positioning 206 a proximal attachment member 110 of the spinal stabilization apparatus 100 above the sacrum bone 14 of the patient 10. The method 200 may further comprise (d) attaching 208 first and second adjustable attachment arms 130, 150 of the spinal stabilization apparatus 100 between the at least one threaded post 170 and the clamp 120 such that each of the first and second adjustable attachment arms 130, 150 extend away from each other on opposite sides of the spine 12 of the patient 10.

In certain optional embodiments, step (d) of the method 200 may further include coupling each of the first and second adjustable attachment arms 130, 150 to the proximal attachment member 110.

In certain optional embodiments, step (d) of the method 200 may further include adjusting a positioning of plurality of arm members 132, 134, 136, 152, 154, 156 of each of the first and second adjustable attachment arms 130, 150 to surround an area of surgical interest 102 and rigidly coupling the plurality of arm members 132, 134, 136, 152, 154, 156 of each of the first and second adjustable attachment arms 130, 150 together to stabilize at least a portion of the spine 12 of the patient 10 positioned between the clamp 120 and the proximal attachment member 110, highlighted by the area of surgical interest 102. In other optional embodiments, the method 200 may further include coupling a plurality of optical reference spheres 190 to each of the first and second adjustable attachment arms 130, 150 such that one optical reference sphere of the plurality of optical reference spheres 190 is positioned at each end of the plurality of arm members 132, 134, 136, 152, 154, 156 of the first and second adjustable attachment arms 130, 150.

In certain optional embodiments, the method 200 may further include coupling a plurality of optical reference spheres 190 along each of the first and second adjustable attachment arms 130, 150.

In certain optional embodiments, the method 200 may further coupling the robot 40 to at least one of the clamp 120 or the proximal attachment member 110.

To facilitate the understanding of the embodiments described herein, a number of terms have been defined above. The terms defined herein have meanings as commonly understood by a person of ordinary skill in the areas relevant to the present invention. Terms such as “a,” “an,” and “the” are not intended to refer to only a singular entity, but rather include the general class of which a specific example may be used for illustration. The terminology herein is used to describe specific embodiments of the invention, but their usage does not delimit the invention, except as set forth in the claims. The phrase “in one embodiment,” as used herein does not necessarily refer to the same embodiment, although it may.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Although embodiments of the present invention have been described in detail, it will be understood by those skilled in the art that various modifications can be made therein without departing from the spirit and scope of the invention as set forth in the appended claims.

This written description uses examples to disclose the invention and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It will be understood that the particular embodiments described herein are shown by way of illustration and not as limitations of the invention. The principal features of this invention may be employed in various embodiments without departing from the scope of the invention. Those of ordinary skill in the art will recognize numerous equivalents to the specific procedures described herein. Such equivalents are considered to be within the scope of this invention and are covered by the claims.

All of the compositions and/or methods disclosed and claimed herein may be made and/or executed without undue experimentation in light of the present disclosure. While the compositions and methods of this invention have been described in terms of the embodiments included herein, it will be apparent to those of ordinary skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

The previous detailed description has been provided for the purposes of illustration and description. Thus, although there have been described particular embodiments of a new and useful invention, it is not intended that such references be construed as limitations upon the scope of this disclosure except as set forth in the following claims.

What is claimed is:

1. A spinal stabilization apparatus for preventing and detecting the unintended movement of a spine of a patient during spinal surgery performed at least partially by a robot, the spinal stabilization apparatus comprising:
a proximal attachment member configured to be positioned above a sacrum bone of the patient;
a distal attachment member configured to be coupled to at least one vertebra of the spine of the patient;
a first adjustable attachment arm configured to be coupled between the proximal attachment member and the distal attachment member, the first adjustable attachment arm further configured to be coupled to a first hip bone of the patient; and
a second adjustable attachment arm configured to be coupled between the proximal attachment member and the distal attachment member and extend away from the first adjustable attachment arm, the second adjustable attachment arm further configured to be coupled to a second hip bone of the patient.

2. The spinal stabilization apparatus of claim 1, wherein:
the first and second adjustable attachment arms are bilaterally symmetric about the spine of the patient when the spinal stabilization apparatus is attached to the spine of the patient.

3. The spinal stabilization apparatus of claim 1, wherein:
the distal attachment member offset from the proximal attachment member by an offset distance; and
each of the first and second adjustable attachment arms are longer than the offset distance.

4. The spinal stabilization apparatus of claim 1, further comprising:
a first threaded post configured to couple the first adjustable attachment arm to an ilium portion of the first hip bone of the patient; and
a second threaded post configured to couple the second adjustable attachment arm to an ilium portion of the second hip bone of the patient.

5. The spinal stabilization apparatus of claim 4, wherein:
the first threaded post includes a first end configured to be attached to the ilium portion of the first hip bone of the patient and a second end configured to be attached to the first adjustable attachment arm; and
the second threaded post includes a first end configured to be attached to the ilium portion of the second hip bone of the patient and a second end configured to be attached to the second adjustable attachment arm.

6. The spinal stabilization apparatus of claim 1, wherein:
the first adjustable attachment arm includes a first arm proximal member, a first arm intermediate member, and a first arm distal member pivotally coupled together, the first arm proximal member configured to be coupled to the proximal attachment member, the first arm distal member configured to be coupled to the distal attachment member; and
the second adjustable attachment arm includes a second arm proximal member, a second arm intermediate member, and a second arm distal member pivotally coupled together, the second arm proximal member configured to be coupled to the proximal attachment member, the second arm distal member configured to be coupled to the distal attachment member.

7. The spinal stabilization apparatus of claim 6, wherein:
the first arm intermediate member is coupled between free ends of the first arm proximal member and the first arm distal member; and
the second arm intermediate member is coupled between free ends of the second arm proximal member and the second arm distal member.

8. The spinal stabilization apparatus of claim 6, wherein:
the first arm proximal member includes a first arm proximal member first end and a first arm proximal member second end;
the first arm proximal member is configured to be coupled to a first threaded post of the spinal stabilization apparatus between the first arm proximal member first end and the first arm proximal member second end, the first threaded post configured to be coupled to an ilium portion of the first hip bone of the patient;
the second arm proximal member includes a second arm proximal member first end and a second arm proximal member second end; and
the second arm proximal member is configured to be coupled to a second threaded post of the spinal stabilization apparatus between the second arm proximal member first end and the second arm proximal member second end, the second threaded post configured to be coupled to an ilium portion of the second hip bone of the patient.

9. The spinal stabilization apparatus of claim 6, further comprising:
a plurality of threaded optical reference spheres, wherein one threaded optical reference sphere of the plurality of threaded optical reference spheres is positioned at each end of each of the first arm proximal member, the first arm distal member, the second arm proximal member, and the second arm distal member.

10. The spinal stabilization apparatus of claim 6, wherein:
the first and second arm intermediate members are telescopic in length.

11. The spinal stabilization apparatus of claim 6, wherein:
each of the first arm proximal member, the first arm intermediate member, and the first arm distal member are configured to be pivotally coupled together using a first plurality of fasteners; and
each of the second arm proximal member, the second arm intermediate member, and the second arm distal member are configured to be pivotally coupled together using a second plurality of fasteners.

12. The spinal stabilization apparatus of claim 1, further comprising:
a plurality of threaded optical reference spheres coupled along each of the first and second adjustable attachment arms, wherein the plurality of threaded optical reference spheres are configured to be monitored for determining if the spine of the patient moves during the course of the spinal surgery.

13. The spinal stabilization apparatus of claim 1, wherein:
the distal attachment member is a spinous process clamp including a vertical post, the vertical post configured to couple to each of the first and second adjustable attachment arms.

14. The spinal stabilization apparatus of claim 1, wherein:
the proximal attachment member includes a proximal robot attachment portion;
the distal attachment member includes a distal robot attachment portion; and
one or more of the proximal robot attachment portion or the distal robot attachment portion is configured to couple to a portion of the robot.

15. The spinal stabilization apparatus of claim 14, further comprising:
at least one strain gauge configured to couple to one or more of the proximal robot attachment portion or the distal robot attachment portion, the at least one strain gauge configured to monitor sprain applied to the spinal stabilization apparatus by at least the robot such that impending movement of the spine of the patient is identifiable.

16. A method of stabilizing a spine of a patient with a spinal stabilization apparatus for spinal surgery performed at least in part by a robot, the method comprising:
(a) coupling at least one threaded post of the spinal stabilization apparatus to one of an ilium portion of a hip bone or a sacrum bone of the patient;
(b) coupling a clamp of the spinal stabilization apparatus to a spinous process of a vertebra of the spine of the patient;
(c) positioning a proximal attachment member of the spinal stabilization apparatus above the sacrum bone of the patient; and
(d) attaching first and second adjustable attachment arms of the spinal stabilization apparatus between the at least one threaded post and the clamp such that each of the first and second adjustable attachment arms extend away from each other on opposite sides of the spine of the patient.

17. The method of claim 16, wherein step (d) further comprises:
coupling each of the first and second adjustable attachment arms to the proximal attachment member.

18. The method of claim 16, wherein step (d) further comprises:
adjusting a positioning of a plurality of arm members of each of the first and second adjustable attachment arms to surround an area of surgical interest; and
rigidly coupling the plurality of arm members of each of the first and second adjustable attachment arms together to stabilize at least a portion of the spine of the patient positioned between the clamp and the proximal attachment member and highlighted by the area of surgical interest.

19. The method of claim 18, further comprising:
coupling a plurality of optical reference spheres to each of the first and second adjustable attachment arms such that one optical reference sphere of the plurality of optical reference spheres is positioned at each end of the plurality of arm members of the first and second adjustable attachment arms.

20. The method of claim 16, further comprising:
coupling a plurality of optical reference spheres along each of the first and second adjustable attachment arms.
